(19) United States
(12) Patent Application Publication
(10) Pub. No.: US 2024/0100517 A1
(43) Pub. Date: Mar. 28, 2024

(54) PCR DETECTION DEVICE AND SYSTEM

(71) Applicants: National Taiwan University, Taipei (TW); Taipei Medical University, Taipei city (TW)

(72) Inventors: HORN-JIUNN SHEEN, Taipei (TW); PEI-KUEN WEI, Taipei City (TW); YU-JUI FAN, Taipei City (TW); PO-HAN JUAN, Taipei (TW); YUNG-YU HUANG, Taipei (TW)

(21) Appl. No.: 17/935,917

(22) Filed: Sep. 27, 2022

(51) Int. Cl. B01L 3/00; B01L 7/00; C12M 1/38

(52) U.S. Cl. CPC B01L 3/5027; B01L 3/50851; B01L 7/52; C12M 1/38; B01L 2300/1827

(57) ABSTRACT

The present disclosure provide a detection device of microfluidic polymerase chain reaction (PCR) and a detection system including the same. This all-in-one device and system may be used to detect at least one biological detection chip, so that can amplify gene fragments at the front-end and detect them at the back-end immediately, decreasing the time required for the analysis, enabling real-time, low-cost, and rapid detection of various viruses, such as EBV and COVID-19, without compromising accuracy or sensitivity.

### BACKGROUND

### Technology Field

The present disclosure relates to a polymerase chain reaction (PCR) detection device and a system comprising the same. Specifically, the present disclosure relates to a microfluidic PCR detection device and a system comprising the same and a detection method of the detection system using the detection device.

### Description of Related Art

In recent years, gene amplification techniques of DNA and mRNA are often used in disease diagnosis, such as polymerase chain reaction (PCR). A further developed techniques are, for example, fluorescence based real-time PCR, real-time PCR (RT-PCR) and quantitative PCR (qPCR). The principle of traditional PCR technology is to amplify DNA molecules by cyclic reactions at different temperatures, and then put the products into a gel electrophoresis device for the detection. However, such detection method is not only time-consuming but also has the disadvantage of low accuracy. For RT-PCR or qPCR, because fluorescent molecules are embedded in the target gene fragment to be amplified, the fluorescent signal is also amplified when the gene fragment is amplified in the amplification stage. At the same time, real-time detection can be obtained by detecting fluorescent signals. However, the disadvantage is that the detection reagents of qPCR are expensive because they contain fluorescent labels.

In order to improve the detection defect of the conventional PCR device, the purpose of the present disclosure is to provide an integrated device of microfluidic PCR and biological detection chip (such as, surface plasmon resonance (SPR) chip) to shorten PCR detection time.

### SUMMARY

The disclosure provides a PCR device comprising: a heating unit comprising a first heating plate and a second heating plate, wherein the first heating plate and the second heating plate are arranged to be parallel and juxtaposed with a spacing d; and a microfluidic substrate comprising a microchannel, wherein the microchannel is configured in the microfluidic substrate with a plurality of isometric parallel back-and-forth cycles. The microfluidic substrate is mounted on the heating unit, and contacts with the first heating plate to form a first heating zone and contacts with the second heating plate to form a second heating zone. The first heating plate applies a first temperature T1 to heat the first heating zone, and the second heating plate applies a second temperature T2 to heat the second heating zone. When T1 is different from T2, the region above the spacing d in the microfluidic substrate forms a third heating zone, and a third temperature T3 formed in the third heating zone is between T1 and T2. Each isometric parallel back-and-forth cycle in the microfluidic substrate passes through the first heating zone, the second heating zone and the third heating zone.

The disclosure also provides a PCR detection device comprising a heating unit comprising a first heating plate and a second heating plate, wherein the first heating plate and the second heating plate are arranged to be parallel and juxtaposed with a spacing d; a microfluidic substrate comprising a microchannel, wherein the microchannel is configured in the microfluidic substrate with a plurality of isometric parallel back-and-forth cycles; and a biological detection chip loaded at the end of the microchannel of the microfluidic substrate in a fluid-communicable manner. The microfluidic substrate is mounted on the heating unit, and contacts with the first heating plate to form a first heating zone and contacts with the second heating plate to form a second heating zone. The first heating plate applies a first temperature T1 to heat the first heating zone, and the second heating plate applies a second temperature T2 to heat the second heating zone. When T1 is different from T2, the region above the spacing d in the microfluidic substrate forms a third heating zone, and a third temperature T3 formed in the third heating zone is between T1 and T2. Each isometric parallel back-and-forth cycle in the microfluidic substrate passes through the first heating zone, the second heating zone and the third heating zone.

The disclosure also provides a PCR detection system comprising a heating unit comprising a first heating plate and a second heating plate, wherein the first heating plate and the second heating plate are arranged to be parallel and juxtaposed with a spacing d; a microfluidic substrate comprising a microchannel, wherein the microchannel is configured in the microfluidic substrate with a plurality of isometric parallel back-and-forth cycles, wherein the microfluidic substrate is mounted on the heating unit and contacts with the first heating plate to form a first heating zone and contacts with the second heating plate to form a second heating zone, wherein the first heating plate applies a first temperature T1 to heat the first heating zone, and the second heating plate applies a second temperature T2 to heat the second heating zone; a biological detection chip loaded at the end of the microchannel of the microfluidic substrate in a fluid-communicable manner; a flow control unit for controlling the flow rate of liquid in the microfluidic substrate; a temperature control unit, which is electrically connected to the heating unit and respectively controls the heating temperatures of the first heating plate and the second heating plate, and a detection unit for detecting the biological detection chip. When T1 is greater than T2, the region above the spacing d in the microfluidic substrate forms a third heating zone, and a third temperature T3 formed in the third heating zone is between T1 and T2.

The disclosure also provides a PCR detection method comprising providing the above PCR detection system; adjusting the temperatures on the first heating plate and the second heating plate in the heating unit for a period of time by the temperature control unit, so that the first heating zone, the second heating zone and the third heating zone are respectively stable at temperatures T1, T2 and T3; and pumping continuously a sample to be tested into the inlet port of the microchannel of the microfluidic substrate for a period of time by the flow control unit, and detecting with the detection unit, and analyzing with an analysis unit.

### BRIEF DESCRIPTION OF THE DRAWINGS

Further features and advantages will become apparent from the following and more particular description of the preferred embodiments of the disclosure, as illustrated in the accompanying drawings, and in which like referenced characters generally refer to the same parts or elements throughout the views, and in which:

FIG. 1 is a schematic drawing of a microfluidic substrate exemplified in the present disclosure.

FIG. 2 is a schematic drawing of a PCR detection device exemplified in the present disclosure.

FIG. 3 is a schematic drawing of a PCR detection system exemplified in the present disclosure.

FIG. 4 is a flow chart of the preparation of the microfluidic substrate exemplified in the present disclosure.

FIG. 5 is another flow chart of the preparation of the microfluidic substrate exemplified in the present disclosure.

FIGS. 6A to 6C are temperature profiles of the heating system of the present disclosure. FIG. 6A shows that the temperatures measured by thermocouple detection in the third heating zone are 72, 66 and 60° C. when the spacing d is 8, 9 and 10 mm, respectively. FIG. 6B shows the temperature stability of the first heating zone, the second heating zone and the third heating zone when the spacing d is 9 mm. FIG. 6C shows three temperature regions used to operate the microfluidic device.

FIG. 7 shows the red shift changes of the resonance wavelength of the detection chip exemplified in the present disclosure before unmodified, after modification with the probe, and after the probe binding to the target nucleic acid.

FIG. 8 shows a comparison of the amplification capabilities of the microfluidic PCR disclosed herein and the conventional PCR machines.

### DETAILED DESCRIPTION OF THE DISCLOSURE

At the outset, it is to be understood that this disclosure is not limited to particularly exemplified materials, architectures, routines, methods or structures as such may vary. Thus, although a number of such options, similar or equivalent to those described herein, can be used in the practice or embodiments of this disclosure, the preferred materials and methods are described herein.

It is also to be understood that the terminology used herein is for the purpose of describing particular embodiments of this disclosure only and is not intended to be limiting.

The detailed description set forth below in connection with the appended drawings is intended as a description of exemplary embodiments of the present disclosure and is not intended to represent the only exemplary embodiments in which the present disclosure can be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of the exemplary embodiments of the specification. It will be apparent to those skilled in the art that the exemplary embodiments of the specification may be practiced without these specific details.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in molecular biology, photology and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

For purposes of convenience and clarity only, directional terms, such as top, bottom, left, right, up, down, over, above, below, beneath, rear, back, and front, may be used with respect to the accompanying drawings. These and similar directional terms should not be construed to limit the scope of the disclosure in any manner.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

Unless defined otherwise, all technical and scientific terms used herein have the same meaning as commonly understood by one having ordinary skill in the art to which the disclosure pertains. Moreover, as used in this specification and the appended claims, the singular forms “a”, “an” and “the” include plural referents unless the content clearly dictates otherwise.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise. This applies regardless of the breadth of the range.

### Microfluidic Substrate

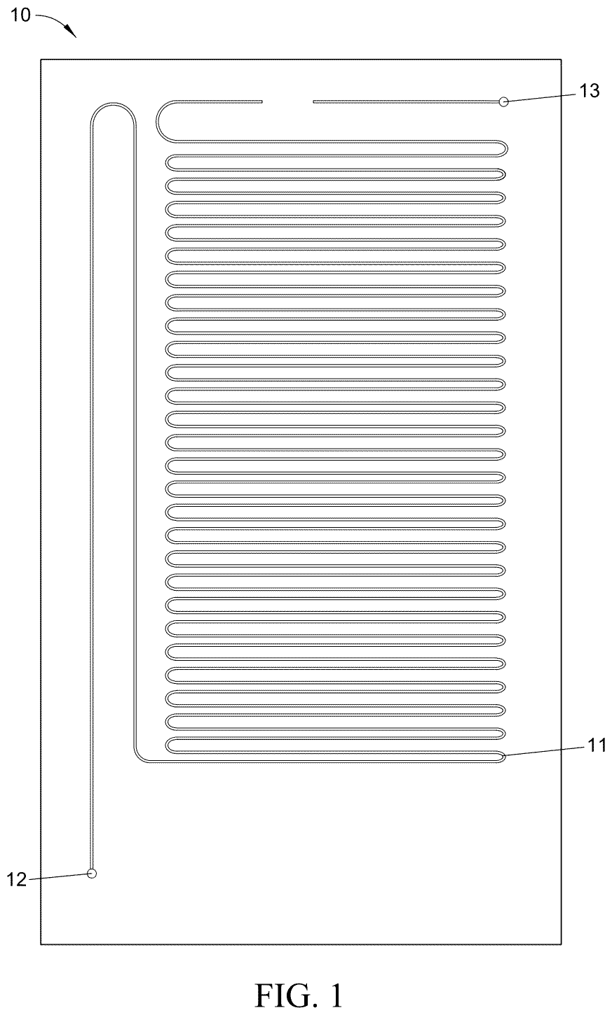

The PCR detection device of the present disclosure comprises a microfluidic substrate, as shown in FIG. 1, comprising at least one microchannel, wherein the at least one microchannel is configured in the microfluidic substrate with a plurality of isometric parallel back-and-forth cycles. In the operation procedure of the PCR detection device, the PCR sample passed through one isometric parallel back-and-forth cycle is equivalent to one thermal cycle by PCR. There is no limit to the number of isometric parallel back-and-forth cycles in the microfluidic substrate, and may be designed according to the number of thermal cycles required to amplify the target in the PCR sample. For example, it may be designed to comprise about 10 to about 50 isometric parallel back-and-forth cycles, preferably about 20 to about 40 cycles, and more preferably about 30 cycles. For example, it may comprise 10, 15, 20, 25, 30, 35, 40, 45 or 50 isometric parallel back-and-forth cycles.

As used herein, the term “isometric parallel back-and-forth” or “isometric parallel back-and-forth cycles” refers to that each cycle path on the microfluidic substrate in the PCR detection device is of equal length and traverses each heating zone so that each cycle may have the same reaction time in each heating zone during PCR amplification. In addition, the paths in the microchannel for the PCR amplification region are substantially parallel to each other to reduce the area of the microfluidic substrate. In other words, for each cycle path, it may be a simple parallel back-and-forth once, or for specific samples, the microchannel may be designed to add several parallel back-and-forth cycles in a specific heating zone, such as to add one or two parallel back-and-forth cycles in the third heating zone, to increase the time required for nucleic acid extension, which is suitable for samples containing longer nucleic acids.

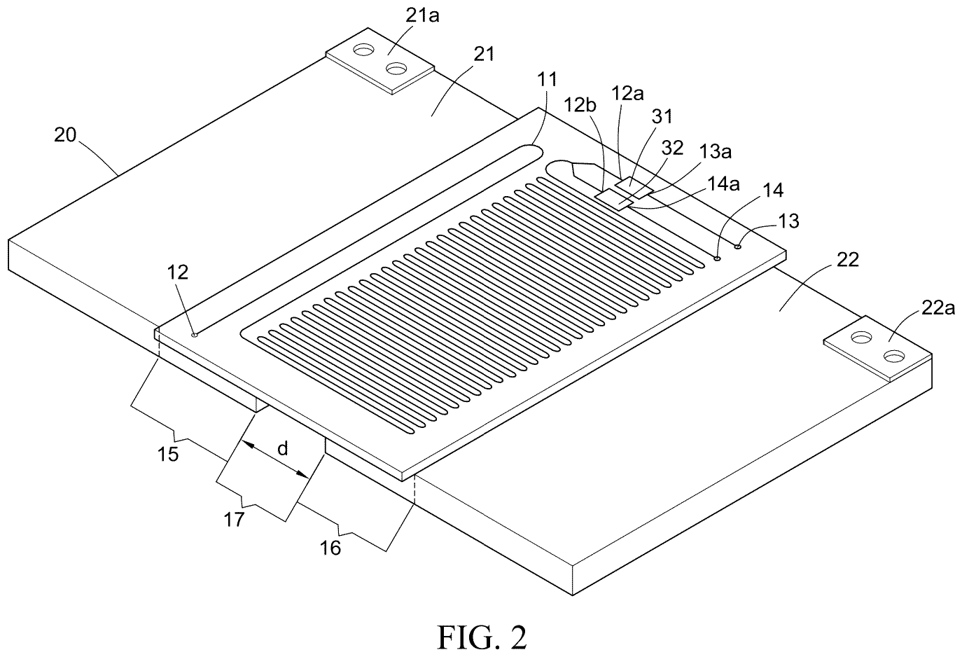

The microchannel in the microfluidic substrate may comprise one inlet port and at least one outlet port. In one embodiment, the end of the microchannel in the microfluidic substrate of the present disclosure may comprise a plurality of outlet port, as shown in FIG. 2, to provide simultaneous detection of multiple biological detection chips, wherein the biological detection chip may be the same or different.

In one embodiment, the microchannel of the present disclosure may be created by using a laser engraving machine to engrave on an appropriate carrier with an appropriate power, speed and focus, and then encapsulating the carrier between two plastic substrates by encapsulation techniques. The plastic substrate is not particularly limited, as long as the plastic substrate that can be used for thermal compression encapsulation to prepare the microchannel can be used in this process, for example, polymethylmethacrylate (PMMA), polycarbonate (PC) and the like can be used. In another embodiment, a laser engraving machine can be used to directly engrave the designed microchannel on two plastic substrates of the same size, and then the two plastic substrates are bonded and encapsulated. The encapsulation technique may, for example, use an organic solvent bonding method, but is not limited thereto. In further embodiment, a laser engraving machine can be used to directly engrave the designed microchannel on one plastic substrate, and then bonded and encapsulated with another plastic substrate of the same size. In one embodiment, the microchannel of the present disclosure may also be prepared by injection molding.

The width, depth and spacing of the microchannels are not particularly limited, and can be optimally designed according to many factors, such as, the biological target to be detected, the type of probe, the fluid flow rate, and the detection chip. In one embodiment, the width of the microchannel of the present disclosure is more than about 50 μm, such as, about 100 to about 400 μm, preferably about 150 to 300 μm. For example, the width of the microchannel may be 100, 150, 200, 250, 300, 350, 400 μm or any size between the above values. In one embodiment, the depth of the microchannel of the present disclosure is more than about 100 μm, such as, about 100 to 200 μm. For example, the depth of the microchannel may be 100, 150, 200 μm or any size between the above values.

There is no particular limitation on the shape of the cross-sectional area of the microchannel, which can be designed and used according to general processing convenience. For example, it can be designed to be square, semicircular, hexagonal, trapezoidal or other cross-sectional shapes.

In one embodiment, the prepared microchannel is encapsulated between two acrylic sheets, and the perimeter of two acrylic sheets is sealed with curing glue. Apiece of acrylic plate is provided with through holes corresponding to the inlet port and the at least one outlet port of the microchannel. Each of the through hole corresponds to the inlet port and the at least one outlet port of the microchannel in a fluid-communicable manner, respectively.

As used herein, the term “fluid-communicable” or “fluid-communicable manner” refers to that the fluid can still flow from channel to channel, channel to through hole, or through hole to through hole after the bonding and encapsulating processes described above.

### Heating Unit

The PCR detection device of the present disclosure comprises a heating unit, which is composed of at least two heating plates. When the heating unit is composed of two heating plates, a first heating plate and a second heating plate are arranged to be parallel and juxtaposed with a spacing d. The above microfluidic substrate may be mounted on the heating unit, that is, across between the first heating plate and the second heating plate, and contacts with the first heating plate to form a first heating zone, and contacts with the second heating plate to form a second heating zone. The region above the spacing d in the microfluidic substrate forms a third heating zone. The first heating plate can apply a first temperature T1 to heat the first heating zone, and the second heating plate can apply a second temperature T2 to heat the second heating zone. When T1 is different from T2, a third temperature T3 formed in the third heating zone will be between T1 and T2.

Since the spacing d can be adjusted by configuring the distance between the first heating plate and the second heating plate, and the contact area between the microfluidic substrate and the first heating plate and the second heating plate can be adjusted, the heating time can be adjusted by configuring the lengths of the path of the microfluidic channel in the first heating zone, the second heating zone and the third heating zone respectively, so as to correspond to the time required for denaturation, primer binding and nucleic acid extension of various PCR samples.

The heating unit in the PCR detection device may be controlled by a temperature control unit, which is electrically connected to the heating unit and can control the heating temperature of the first heating plate and the second heating plate respectively. In addition, the spacing d may be in the range of about 1 mm to about 10 mm. Therefore, by controlling the heating temperatures of the first heating plate and the second heating plate and the range of the spacing d, T1, T2 and T3 can be easily fixed at desired temperatures. In one embodiment, when the PCR sample is a salt-free sample, the temperatures are preferably about 80° C. to 90° C. for T1 and about 55° C. to about 65° C. for T2 respectively, and the spacing d is in the range of about 1 mm to about 6 mm. In another embodiment, when the PCR sample is a salt-containing sample, the temperatures are preferably about 90° C. to about 100° C. for T1 and about 55° C. to about 65° C. for T2 respectively, and the spacing d is in the range of about 6 mm to about 10 mm.

### PCR Detection Device and Detection System

In one embodiment, the present disclosure provides a PCR detection device, as shown in FIG. 2, which at least comprises a microfluidic substrate 10, a heating unit 20, a first biological detection chip 31 and a second biological detection chip 32. The microfluidic substrate 10 comprises a microchannel 11 comprising an inlet port 12 and two outlet ports 13 and 14. The heating unit 20 comprises a first heating plate 21 and a second heating plate 22, wherein the first heating plate 21 and the second heating plate 22 are arranged to be parallel and juxtaposed with a spacing d. The first heating plate 21 and the second heating plate 22 respectively comprise temperature control ports 21a and 22a, which can be electrically connected to the temperature control unit. The biological detection chips 31 and 32 are disposed near the ends of microchannel 11 on the microfluidic substrate 10. The two ends of the first biological detection chip 31 are respectively loaded on the chip microchannel connection ports 12a and 13a at the ends of the microchannel 11 in a fluid-communicable manner. The two ends of the second biological detection chip 32 are respectively loaded on the chip microchannel connection ports 12b and 14a at the ends of the microchannel 11 in a fluid-communicable manner.

The microfluidic substrate 10 is mounted on the heating unit 20 and contacts with the first heating plate 21 to form a first heating zone 15, and with the second heating plate 22 to form a second heating zone 16. On the microfluidic substrate 10, the region between the first heating zone 15 and the second heating zone 16 is a third heating zone 17. The heating area of the third heating zone 17 may be adjusted by configuring the distance between the first heating plate 21 and the second heating plate 22. In addition, the heating area of the first heating zone 15 and the second heating zone 16 can be adjusted by configuring the position where the microfluidic substrate 10 is placed on the heating unit 20. In FIG. 2, for example, when the spacing d is not changed, the microfluidic substrate 10 is placed to the left, then a larger first heating area 15 and a smaller second heating area 16 can be obtained.

Since the microchannel in the microfluidic substrate of the present disclosure has a consistent diameter, the time required for the sample to flow through a unit spacing in the microchannel can be calculated from the flow rate of the sample. Therefore, the PCR detection device of the present disclosure can simply and accurately determine the areas of the three heating zones through the above adjustment, thereby adjusting the heating time of the sample. Specifically, in addition to controlling the time for the sample to pass through the three heating zones by the amount of sample pumped into the microchannel per unit time, the time of the sample to flow through the three heating zones can be adjusted more optimally by adjusting the position where the microfluidic substrate 10 is placed on the heating unit 20 and adjusting the spacing d, so as to meet the temperature and time required for denaturation, annealing/primer bonding, and extension steps required for PCR amplification of different biological samples. In other words, the PCR detection device and detection system of the present disclosure have wider application field.

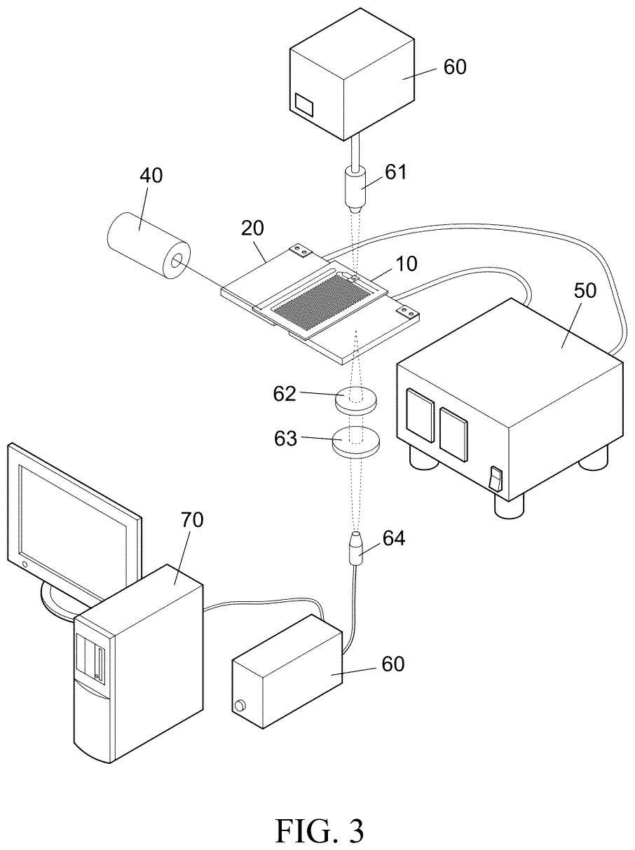

In one embodiment, the present disclosure provides a PCR detection system. As shown in FIG. 3, in addition to the PCR detection device described above, the PCR detection system further comprises a flow control unit 40, a temperature control unit 50, detection unit 60 and an analysis unit 70.

The flow control unit 40 of the present disclosure may be a micropump or other similar device. By using a microtube, the inlet port and the outlet port of the micropump can be connected to a sample bottle and the inlet port 12 of the microchannel (as shown in FIG. 2) in a fluid-communicable manner. The flow control unit 40 may have an independent control unit to control the feed rate, or may be integrated into other units, such as an analysis unit 70.

The temperature control unit 50 of the present disclosure is electrically connected to the heating unit 20. The temperature control unit 50 can independently control at least two heating units, preferably three to four heating units. The temperature control unit 50 is electrically connected to a temperature control ports 21a and 22a respectively on the first heating plate 21 and the second heating plate 22 in the heating unit 20 (as shown in FIG. 2) for respectively and independently controlling the temperatures of the first heating plate 21 and the second heating plate 22.

The detection unit 60 of the present disclosure is used for detecting the results of the biological detection chip, such as 31 and 32 as shown in FIG. 2, on the microfluidic substrate 10, and the instruments of the detection unit 60 can be changed according to the optimal detection method of the biological detection chip. For example, the objective lens 61, the polarizer 62, the lens 63 and/or the optical fiber 64 may be further combined for a specific detection sample.

The analysis unit 70 of the present disclosure is electrically connected to the detection unit 60 for analyzing the results obtained from the detection unit 60. The analysis unit 70 can perform analysis and comparison with various analysis software and databases in itself, on the Internet or in the cloud, and provide various comparative analysis results, such as various analysis graphs. In one embodiment, the analysis unit 70 of the present disclosure may integrate with other units in the system. For example, the analysis unit 70 may integrate with the flow control unit 40, the temperature control unit 50 and the detection unit 60 to be a single control-detection-analysis unit.

### Detection Method

The present disclosure provides a PCR detection method comprising:
providing the PCR detection system described above,
adjusting the temperatures on the first heating plate 21 and the second heating plate 22 in the heating unit 20 for a period of time by the temperature control unit 50, so that the first heating zone 15, the second heating zone 16 and the third heating zone 17 are respectively stable at temperatures T1, T2 and T3,
pumping continuously a sample to be tested into the inlet port 12 of the microchannel 11 of the microfluidic substrate 10 for a period of time by the flow control unit 40, detecting with the detection unit 60, and analyzing with the analysis unit 70.

The biological detection chip can be selected according to the sample to be tested and assembled into the system to perform the PCR detection method of the present disclosure. For example, probes for detecting the nucleic acid can be selected or designed according to the type of nucleic acid to be detected, and attached to the nanochannels of the biological detection chip by known techniques. The minimum amount of detectable binding to the probe on the biological detection chip can be achieved by the amplification in a plurality of isometric parallel back-and-forth cycles after a micro amount of nucleic acid to be tested passes through the microfluidic channel. Then, it is detected by the detection unit 60 and analyzed by the analysis unit 70.

### EXAMPLES

### Example 1: Materials and Methods

1. Preparation of Test Samples

The present disclosure uses Epstein-Barr virus (EBV)-related nucleic acids and proteins as test samples. EBV is a human herpesvirus associated with various cancers, including nasopharyngeal carcinoma, Burkitt's lymphoma, and Hodgkin's lymphoma. Six nuclear antigens and three membrane proteins cooperate to induce the proliferation and transformation of EBV-infected cells. Among these, latent membrane protein 1 (LMP1) is expressed in most EBV-related malignancies, and it is recognized as a prognostic biomarker in those diseases.

Test samples were acquired from nasopharyngeal cancer patients, and the samples were preprocessed to obtain LMP1 DNA. Further, LMP1 DNA was amplified with the PCR and analyzed by electrophoresis. In this research, primers and the template were modified, and total length of LMP1 DNA was 311 base pairs (bp). Details of the primer and LMP1 DNA were as following Table 1:

TABLE 1
LMP1 template and primer designs

| | | |
|---|---|---|
| LMP1 | Template | 5′-CCATGACCCGCTGCCTCATAACCCTAGCGACTCTGCT |
| | | GGAAATGATGGAGGCCCTCCAAAATTGACGGAAGAGGT |
| | | TGAAAACAAAGGAGGTGACCGGGGCCCGCCTTCGATGA |
| | | CAGACGGTGGCGGCGGTCATCCACACCTTCCTACACTGC |
| | | TTTTGGGTACTTCTGGTTCCGGTGGAGATGATGACGACC |
| | | CCCACGGCCCAGTTCAGCTAAGCTACTATGACTAACCTT |
| | | TCTTTACTTCTAGGCATTACCATGTCATAGGCTTGCCTGA |
| | | CTGACTCTCCCTCCATTTACTGGGAATGCCTTAGCTAATC |
| | | A-3′ |
| | Forward | 5′-AGC GAC TCT GCT GGA AAT GAT-3′ |
| | Reverse | 5′-TGA TTA GCT AAG GCA TTC CCA-3′ |

2. DNA Extraction

The specimens used were derived from 4 types of EBV-positive cell lines and 1 type of EBV-negative cell line. HKNPC-C43, or NPC43, is an EBV-positive nasopharyngeal carcinoma cell line (a gift from Prof. George Sai Wah Tsao); Akata, P3HR1, and NAMALWA are EBV-positive Burkitt's lymphoma cell lines. DB is an EBV-negative diffuse large cell lymphoma cell line. DNA was extracted from these cells using commercial kit (AxyPrep Multisource Genomic DNA Miniprep Kit, 50 prep). The following procedures are referred to the manufacturer's protocols. After extraction, samples were collected and measured with NanoDrop (Thermo Scientific™), to identify the DNA concentration and purity.

3. PCR Parameters

The PCR solution (Taq PCR Master Mix) was purchased from Qiagen (Germantown, MD, USA), including the forward and reverse primers, the DNA template, and nuclease-free water. In the device, each reaction used 50 μl of a mixed solution, which included 25 μl of master mix, 1 μl of 1011M forward primer, 1 μl of 10 μM reverse primer, 1 μl of 10 μg/ml of the DNA template, and 22 μl of nuclease-free water.

The parameters of the traditional PCR machine, as the control group, were as following Table 2.

TABLE 2
The reaction parameters of the traditional PCR machine

| | | | | | |
|---|---|---|---|---|---|
| | Initial denaturation | 95° | C. | 5 | min |
| 30 | Denaturation | 95° | C. | 30 | s |
| cycles | Annealing/ | 60° | C. | 50 | s |
| | Primer bonding | | | | |
| | Extension | 72° | C. | 50 | s |
| | Final extension | 72° | C. | 10 | min |
| | Storage | 4° | C. | 10 | min |

4. Nonaslit Surface Plasmon Resonance (SPR) Chip

The gold-capped nanoslit chip were prepared by the method proposed in Chuang, C. S., Wu, C. Y, Juan, P. H., Hou, N.C., Fan, Y-J., Wei, P. K., Sheen, H. J., 2020. Analyst 145, 52-60, incorporated herein by reference in the entirety. The defined nanoslit structure (with a 500-nm interval and an 80-nm width) was precisely etched on the silicon wafer and then transferred onto a nickel-cobalt (Ni—Co) alloy mold through the electrode position method. Afterward, the nanostructure on the Ni—Co mold was imprinted onto a polycarbonate (PC) film by hot-embossing nanoimprinting lithography (at a temperature of 165° C. and a working pressure of 690 kPa) according to the method proposed in Lee, K. L., Chen, P. W., Wu, S. H., Huang, J. B., Yang, S. Y, Wei, P. K., 2012. ACS Nano 6, 2931-2939; and Lee, K. L., You, M. L., Tsai, C. H., Lin, E. H., Hsieh, S. Y, Ho, M. H., Hsu, J. C., Wei, P. K., 2016, incorporated herein by reference in the entirety. Finally, the gold was deposited (with a deposition time of 70 s, a working pressure of 4×10⁻⁶ kPa, air flow of 50 standard cm3/min (sccm), and power of 0.06 kW) onto the nanoslit PC film using a direct current sputtering system (Ulvac, Methuen, MA, USA). To integrate the microfluidic PCR with the nanoslit SPR chip, the SPR chip was bonded to the end of the PCR microchannel in a fluid-communicable manner by using the heat-resistant double-sided tape which had a chamber opening.

5. Surface Modification of the SPR Chip

To detect the tested LMP1 gene, the SPR chip in the microfluidic device was further modified with an LMP1 DNA probe (5 ′-GTCATAGTAGCTTAGCTGAACTGGGCCGT-3′).

A cysteamine solution (100 μg/ml) was allowed to flow into the device prepared above and was incubated for 2 h, which allowed the cysteamine to bind to the gold-capped surface of the SPR chip via thiol groups. After washing with deionized (DI) water, the LMP1 probe was pumped into the device and kept for different time intervals for electrostatic adsorption of the LMP1 DNA probe onto the SPR chip. Eventually, the device was washed with DI water to complete the surface modification.

6. Optical Measuring Device

An optical setup for measuring the transparent type of resonant spectrum of the gold-capped nanoslit SPR chip was constructed. When broadband white light passes through the nanoslit SPR chip, a polarizer at the other side of SPR chip filters out the resonant wavelength in the transverse magnetic (TM) direction. The transmitted light was collected with an optical fiber via a fiber lens and then measured with a spectrometer (B&W Tek, Newark, DE, USA).

7. Analysis of SPR Data

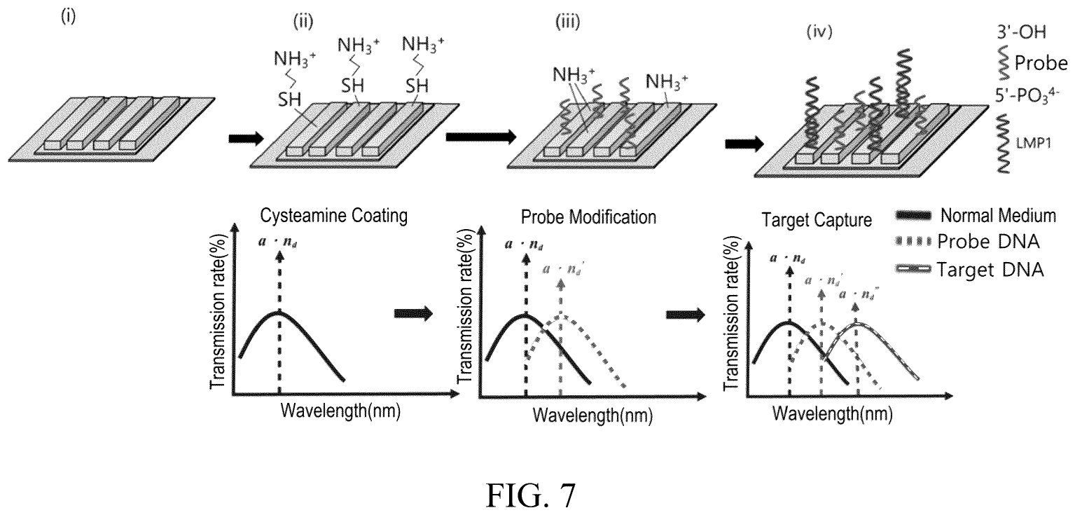

The sensing mechanism is illustrated in FIG. 7. The resonant wavelength (λ) in the TM direction can be expressed as

λ=a·dp; (1)

where a is the environmental refractive index nearby gold-capped nanoslit, and dp is the period of the nanoslit.

Initially, the nanoslit SPR chip was immersed in water, and a was equal to 1.33 at this time. When the LMP1 probe was modified onto the gold-capped nanoslit surface, the refractive index increased, and the resonant wavelength was red-shifted. Subsequently, when sensing the LMP1 target and binding onto the probe, the resonant wavelength was red-shifted again because of an increase in the refractive index. To quantify the red-shift, the spectral centroid method was used for spectrum processing to locate the resonant wavelength. The calculated resonant wavelength at different time intervals was recorded every 2 s and plotted in a time-dependent diagram for observation of changes in the resonance wavelength at different DNA-binding stages. The red-shift value was evaluated by subtracting the average wavelengths before and after DNA was bound to the nanoslit SPR chip.

### Example 2: Preparation of the Microfluidic Substrate

The microchannel pattern (as shown in FIG. 1) was designed by using AutoCAD (Autodesk, USA) software. The microchannel was designed as a single path having a plurality of isometric parallel back-and-forth cycles, and the microchannel was created by laser scribing technology.

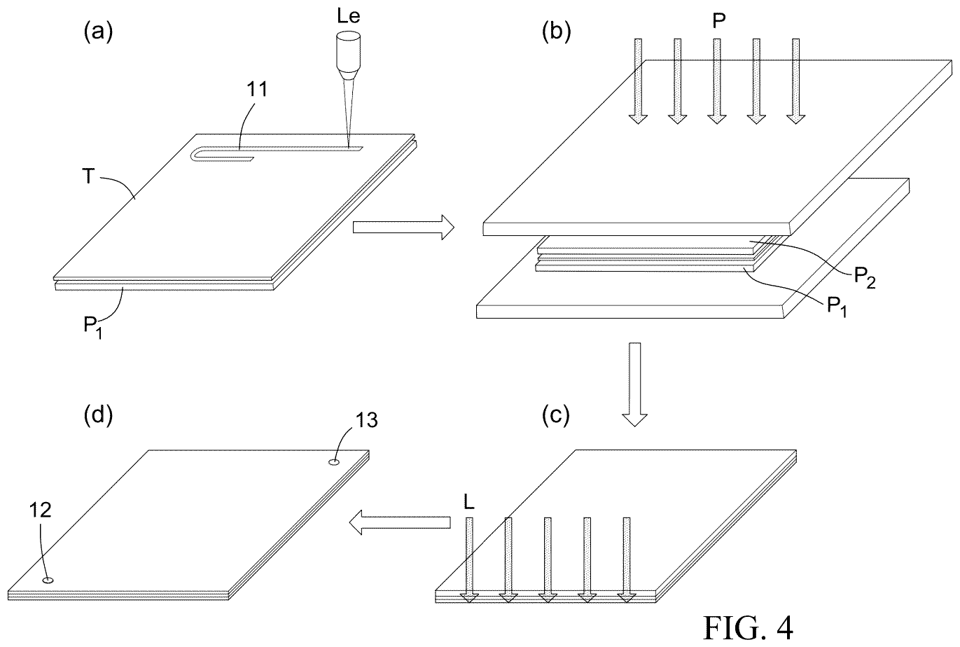

Referring to the preparation process shown in FIG. 4, a carrier is used to prepare a microfluidic substrate. First, the heat-resistant double-sided tape T (400 μm in thickness) as a carrier was adhered to the first acrylic sheet P1 and then removed by a laser beam of the laser engraving machine Le with the designed pattern to create a microchannel (300 μm in width) (FIG. 4(a)). The single path having a plurality of isometric parallel back-and-forth cycles (1100 μm in period) provided different regions for corresponding PCR temperatures, which can heat or cool the sample 30 times between about 95° C. and about 60° C. Then, the second acrylic sheet P2 with a reserved inlet and outlet hole opening was bound to the other side of the patterned tape by the way of aligning respectively the inlet and outlet with the start and end points of the microchannel. Two sheets are pressed together with uniform pressure P (100 kPa) (FIG. 4(b)) and irradiated with UV illumination L (10 min) (FIG. 4(c)) to cure and seal the glue, and then the microfluidic substrate can be obtained (FIG. 4(d)).

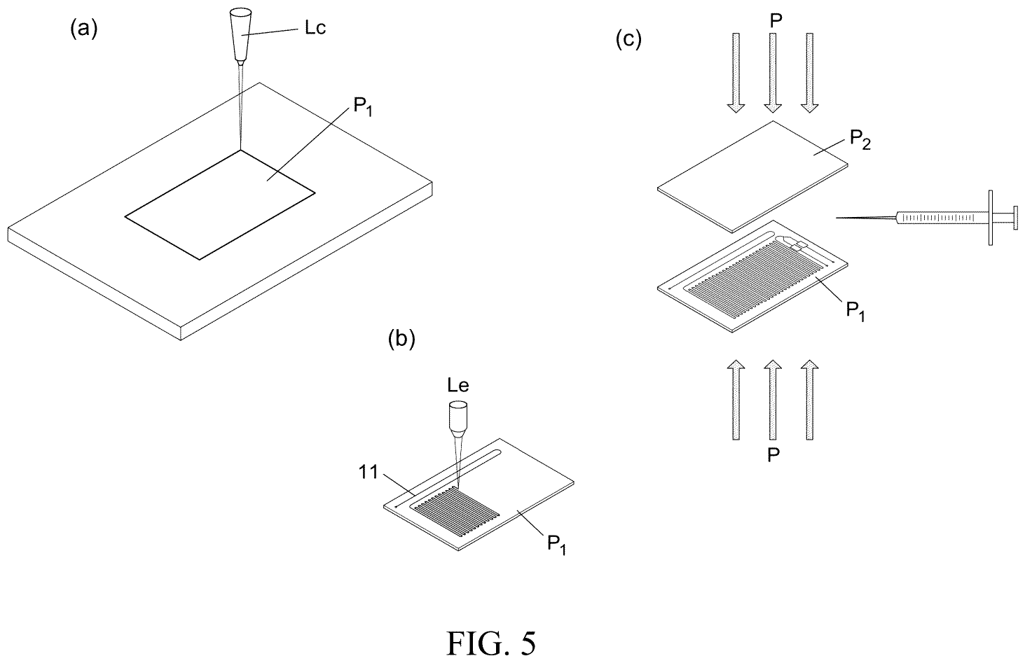

Referring to the preparation process shown in FIG. 5, a microfluidic substrate is prepared without carrier. First, two pieces of the same size (length 87 mm, width 50 mm) of the first acrylic sheet P1 and the second acrylic sheet P2 were cut out by a laser cutter (FIG. 5(a)). The first acrylic sheet P1 was engraved directly by using the laser engraving machine Le (CNC engraving machine, EGX-400, Roland) with the designed microfluidic pattern (FIG. 5(b)). The microchannel was designed as a single path with a width of 300 μm and a depth of 100 which had a plurality of isometric parallel back-and-forth cycles and provided different regions for corresponding PCR temperatures for heating or cooling the sample 30 times between 95° C. and 60° C. Then, the first acrylic sheet P1 and the second acrylic sheet P2 were encapsulated as microchannel substrates by organic solvent bonding. That is, a mixture of 20% acetone and 80% alcohol was prepared as an organic solvent and coated on the acrylic substrate without microchannel. The two acrylic sheets were closely attached, and finally put into the hot embossing machine. The target temperature was set to 60° C., and the pressure P was 5 kg/cm2. Under these conditions, it was cured for about 3 min (FIG. 5(c)). After cooling down, the encapsulation of the microfluidic substrate was completed.

The size of the detection chamber for setting the detection chip was 10×10 mm square in line with the chip, and the interior was designed as a polygon to reduce the generation of air bubbles and ensure that the fluid fills the detection chamber stably. Similar to the above process, the heat-resistant double-sided tape with a thickness of 400 μm is cut by a laser engraving machine.

### Example 3: Heating Device Settings

When the microfluidic device performs the PCR procedure, the three temperature zones for denaturation, primer binding and extension of PCR samples were respectively set as: T1 was 95° C. for denaturation; T2 was 60° C. for annealing/primer bonding; and T3 was 65° C. for extension and detection.

First, the first heating plate and the second heating plate were two aluminum blocks with an electric heating film, which separately defined the first heating zone and the second heating zone as 95° C. and 60° C., while the third heating zone at 65° C. in the middle of the device was maintained by adjusting the spacing d and the heating plates on both sides. A proportional integral derivative (PID) controller was used to adjust the current to achieve the desired temperature, and the resistance temperature detector attached to the heating plates sent the signal back to the PID controller to stabilize the heating process. The actual temperature distribution of the device was examined using a thermographic camera (Flir, Wilsonville, OR, United States, USA).

### Example 4: Temperature Distribution of the Microfluidic Substrate

A thermographic camera and thermocouple were used to determine the stability of the temperature distribution in the microfluidic device.

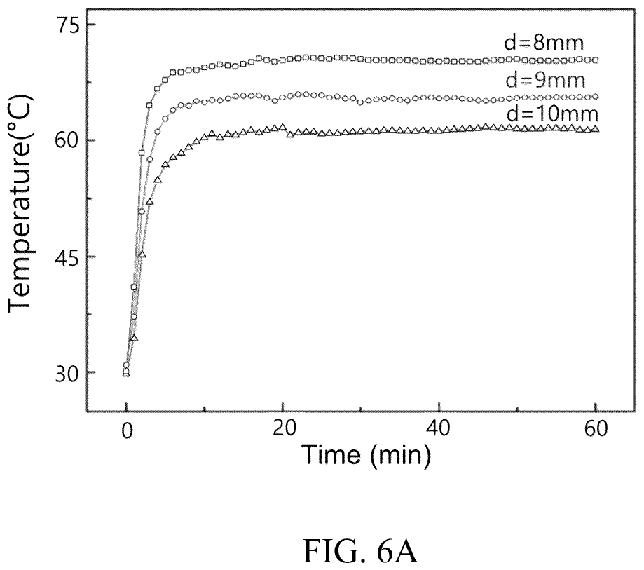

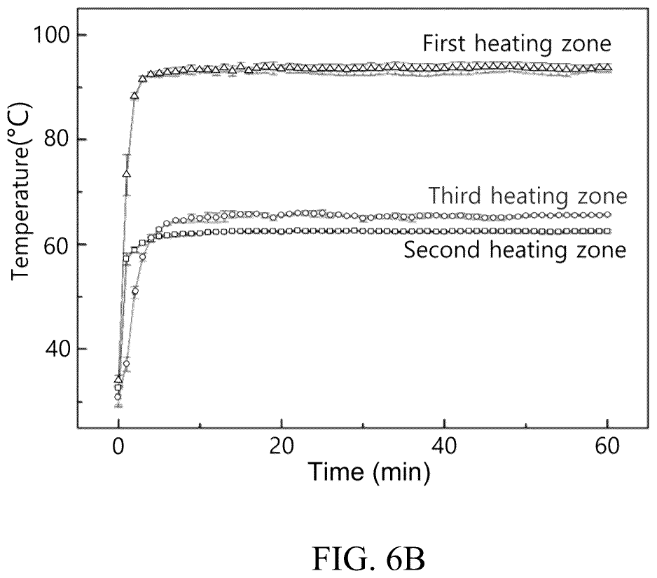

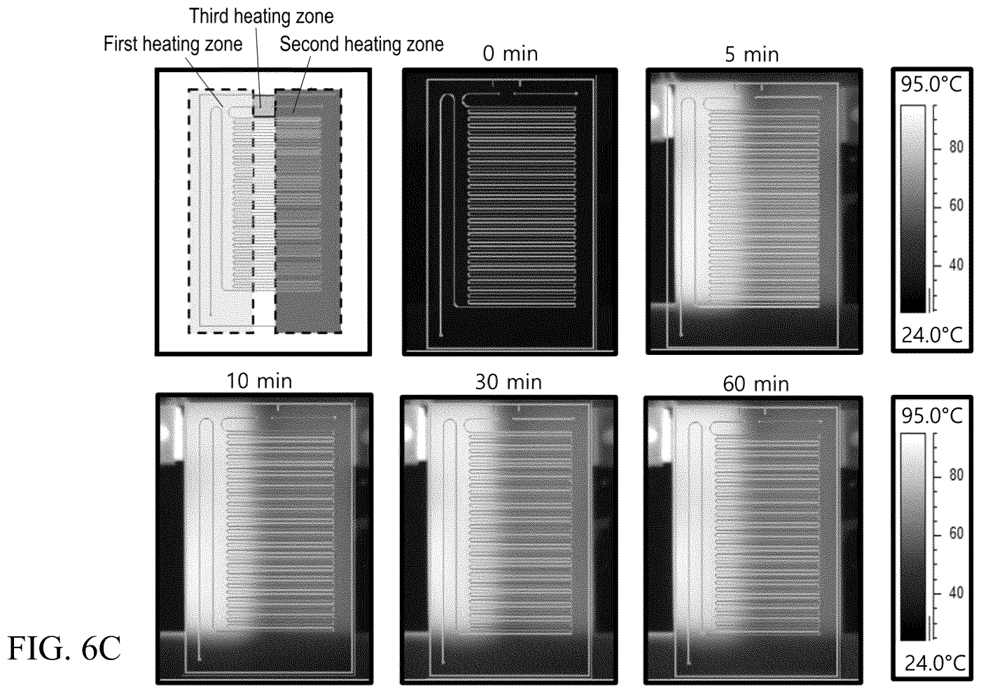

First, the thermocouple monitored the temperature of each heating zone when the spacing d between the two heating plates changed. The temperatures were recorded each minute, and 8, 9, and 10 cm were applied as the spacing d. The results showed that temperatures were stable within 10 min (FIG. 6A). The smaller spacing d had a higher temperature in the third heating zone.

For the microfluidic PCR, reagents need thermal cycling between 95° C. and 60° C. for DNA amplification, while for the SPR chip, the temperature should be kept around 63.7° C., which is the melting temperature (Tm) of the probe, for DNA detection. Therefore, the spacing d 9 cm was used. The thermocouple monitored the temperatures in the regions for denaturation (the first heating zone), primer binding (the second heating zone), and extension and detection (the third heating zone) (FIG. 6B).

The results revealed that desired temperatures were achieved in the device after heating for 5 min (FIG. 6C). A homogeneous temperature distribution was observed in the microfluidic PCR region which was maintained for at least 1 hour. Also, the third heating zone was kept at 65° C. when the heating plates were 10 mm apart.

### Example 5: Amplification Capacity of the Microfluidic PCR Device

Gel electrophoresis was used to confirm the products of the microfluidic PCR device of the present disclosure under different flow rates. First, the PCR solution containing LMP1 DNA at an initial concentration of 10 μg/mL was pumped into the microfluidic PCR, and the syringe pump was set to different flow rates, that is, 3, 4, 5, 6, and 7 μl/min. Then, the products were analyzed by gel electrophoresis.

As a result, under flow rates of 3, 4, and 5 μl/min, the fluorescent signal of the gel electrophoresis appeared in the correct position (300 bp), indicating that the microfluidic device of the present disclosure could successfully amplify DNA under this flow condition. In contrast, with flow rates of 6 and 7 μl/min, the fluorescent signal was not seen, indicating that the flow rate was too fast to produce the product, resulting in an undetectable low concentration. These results revealed that the microfluidic PCR device of the present disclosure could successfully amplify LMP1 DNA.

### Example 6: Detection Capability of the Microfluidic PCR Device Integrated with the Biological Detection Chip

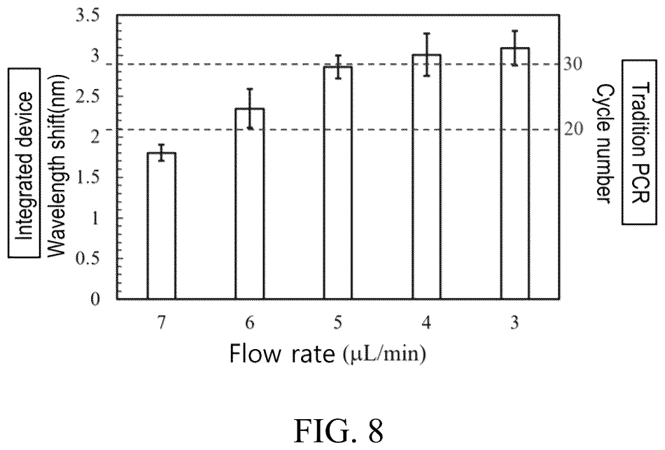

To confirm the detection capability of the integrated device, the red-shift from the PCR detection device of the present disclosure with 10 min of incubation time was recorded under different flow rates, then juxtaposed with the red-shift from the product amplified by a traditional PCR machine to evaluate the detection capability of the integrated device (FIG. 8). The red-shift from the device with flow rates of 3 and 4 μl/min was similar to the traditional machine with 30 cycles, and the red-shift from the device remained at a plateau, which indicates that the SPR sensor had reached its maximum capacity for detection. In contrast, the red-shift of the device with flow rates of 6 and 7 μl/min was lower than that of the traditional machine with 30 cycles. In particular, the red-shift from the device with a 7 μl/min flow rate was even less than that of the traditional machine with 20 cycles.

On the other hand, at a flow rate of 5111/min, the PCR detection device of the present disclosure exhibited a similar amplification capability as the traditional machine, and the reaction time was significantly reduced. The reaction time for the PCR detection device of the present disclosure only took 36 min, but the traditional machine required 105 min to amplify 30 cycles. Therefore, 5 μl/min can be regarded as an optimal reaction flow rate for the PCR detection device of the present disclosure under the conditions of this example.

In addition, the SPR chip could express the corresponding red-shift at different flow rates before it reached its maximum capacity, which suggests that the SPR chip can detect LMP1 DNA and perform quantitative analyses to a certain extent based on the concentration.

### Efficacy

Compared to a traditional PCR machine, the PCR detection device of the present disclosure can significantly reduce the amount of sample required. Also, because the contact area between the PCR solution and heat source is increased, temperature control is more efficient. Using the PCR detection device of the present disclosure can reduce the overall PCR amplification time. After amplification, target DNA is captured by the DNA probe used on the gold nanoslit and sensitively detected by the SPR method. In brief, the device of the present disclosure can amplify, such as LMP1 gene fragments at the front-end and perform detection at the back-end, accelerating the PCR speed, and also has high specificity and sensitivity for detecting the target DNA.

### ENUMERATED EMBODIMENTS

The following exemplary embodiments are provided, the numbering of which is not to be construed as designating levels of importance.

Embodiment 1 provides a PCR device comprising
a heating unit comprising a first heating plate and a second heating plate, wherein the first heating plate and the second heating plate are arranged to be parallel and juxtaposed with a spacing d, and
a microfluidic substrate comprising a microchannel, wherein the microchannel is configured in the microfluidic substrate with a plurality of isometric parallel back-and-forth cycles,
wherein the microfluidic substrate is mounted on the heating unit, and contacts with the first heating plate to form a first heating zone and contacts with the second heating plate to form a second heating zone,
wherein the first heating plate applies a first temperature T1 to heat the first heating zone, and the second heating plate applies a second temperature T2 to heat the second heating zone,
wherein when T1 is different from T2, the region above the spacing d in the microfluidic substrate forms a third heating zone, and a third temperature T3 formed in the third heating zone is between T1 and T2,
wherein each isometric parallel back-and-forth cycle in the microfluidic substrate passes through the first heating zone, the second heating zone and the third heating zone.

Embodiment 2 provides the PCR device of Embodiment 1, wherein the spacing d is in the range of 1 to 10 mm.

Embodiment 3 provides the PCR device of Embodiment 1, wherein the microchannel in the microfluidic substrate comprises one inlet port and at least one outlet port.

Embodiment 4 provides a PCR detection device comprising
a heating unit comprising a first heating plate and a second heating plate, wherein the first heating plate and the second heating plate are arranged to be parallel and juxtaposed with a spacing d,
a microfluidic substrate comprising a microchannel, wherein the microchannel is configured in the microfluidic substrate with a plurality of isometric parallel back-and-forth cycles, and
a biological detection chip loaded at the end of the microchannel of the microfluidic substrate in a fluid-communicable manner,
wherein the microfluidic substrate is mounted on the heating unit, and contacts with the first heating plate to form a first heating zone and contacts with the second heating plate to form a second heating zone,
wherein the first heating plate applies a first temperature T1 to heat the first heating zone, and the second heating plate applies a second temperature T2 to heat the second heating zone,
wherein when T1 is different from T2, the region above the spacing d in the microfluidic substrate forms a third heating zone, and a third temperature T3 formed in the third heating zone is between T1 and T2,
wherein each isometric parallel back-and-forth cycle in the microfluidic substrate passes through the first heating zone, the second heating zone and the third heating zone.

Embodiment 5 provides the PCR detection device of Embodiment 4, wherein the biological detection chip is a surface plasmon resonance chip.

Embodiment 6 provides the PCR detection device of Embodiment 4, which further comprises a flow control unit for controlling the flow rate of liquid in the microfluidic substrate.

Embodiment 7 provides the PCR detection device of Embodiment 4, which further comprises a temperature control unit, wherein the temperature control unit is electrically connected to the heating unit and respectively controls the heating temperatures of the first heating plate and the second heating plate.

Embodiment 8 provides the PCR detection device of Embodiment 4, wherein the spacing d is in the range of 1 to 10 mm.

Embodiment 9 provides the PCR detection device of Embodiment 4, wherein the spacing d is in the range of 1 to 6 mm for a salt-free sample.

Embodiment 10 provides the PCR detection device of Embodiment 4, wherein the spacing d is in the range of 6 to 10 mm for a salt-containing sample.

Embodiment 11 provides the PCR detection device of Embodiment 4, wherein the temperature of the first heating plate is controlled in the range of 80° C. to 100° C. when T1 is greater than T2.

Embodiment 12 provides the PCR detection device of Embodiment 4, wherein the temperature of the second heating plate is controlled in the range of 55° C. to 65° C. when T1 is greater than T2.

Embodiment 13 provides the PCR detection device of Embodiment 4, wherein the temperature of the first heating plate is controlled in the range of 80° C. to 90° C. and the temperature of the second heating plate is controlled in the range of 55° C. to 65° C. for a salt-free sample.

Embodiment 14 provides the PCR detection device of Embodiment 4, wherein the temperature of the first heating plate is controlled in the range of 90° C. to 100° C. and the temperature of the second heating plate is controlled in the range of 55° C. to 65° C. for a salt-containing sample.

Embodiment 15 provides the PCR detection device of Embodiment 4, wherein the microchannel in the microfluidic substrate comprises one inlet port and at least one outlet port.

Embodiment 16 provides a PCR detection system comprising
a heating unit comprising a first heating plate and a second heating plate, wherein the first heating plate and the second heating plate are arranged to be parallel and juxtaposed with a spacing d,
a microfluidic substrate comprising a microchannel, wherein the microchannel is configured in the microfluidic substrate with a plurality of isometric parallel back-and-forth cycles, wherein the microfluidic substrate is mounted on the heating unit and contacts with the first heating plate to form a first heating zone and contacts with the second heating plate to form a second heating zone, wherein the first heating plate applies a first temperature T1 to heat the first heating zone, and the second heating plate applies a second temperature T2 to heat the second heating zone,
a biological detection chip loaded at the end of the microchannel of the microfluidic substrate in a fluid-communicable manner,
a flow control unit for controlling the flow rate of liquid in the microfluidic substrate,
a temperature control unit, which is electrically connected to the heating unit and respectively controls the heating temperatures of the first heating plate and the second heating plate, and
a detection unit for detecting the biological detection chip,
wherein when T1 is greater than T2, the region above the spacing d in the microfluidic substrate forms a third heating zone, and a third temperature T3 formed in the third heating zone is between T1 and T2.

Embodiment 17 provides the PCR detection system of Embodiment 16, wherein the biological detection chip is a surface plasmon resonance chip.

Embodiment 18 provides the PCR detection system of Embodiment 16, wherein the spacing d is in the range of 1 to 10 mm.

Embodiment 19 provides the PCR detection system of Embodiment 16, wherein the spacing d is in the range of 1 to 6 mm for a salt-free sample.

Embodiment 20 provides the PCR detection system of Embodiment 16, wherein the spacing d is in the range of 6 to 10 mm for a salt-containing sample.

Embodiment 21 provides the PCR detection system of Embodiment 16, wherein the temperature of the first heating plate is controlled in the range of 80° C. to 100° C. when T1 is greater than T2.

Embodiment 22 provides the PCR detection system of Embodiment 16, wherein the temperature of the second heating plate is controlled in the range of 55° C. to 65° C. when T1 is greater than T2.

Embodiment 23 provides the PCR detection system of Embodiment 16, wherein the temperature of the first heating plate is controlled in the range of 80° C. to 90° C. and the temperature of the second heating plate is controlled in the range of 55° C. to 65° C. for a salt-free sample.

Embodiment 24 provides the PCR detection system of Embodiment 16, wherein the temperature of the first heating plate is controlled in the range of 90° C. to 100° C. and the temperature of the second heating plate is controlled in the range of 55° C. to 65° C. for a salt-containing sample.

Embodiment 25 provides the PCR detection system of Embodiment 16, wherein the microchannel in the microfluidic substrate comprises one inlet port and at least one outlet port.

Embodimen 26 provides a PCR detection method comprising
providing the PCR detection system of any one of Embodiments 16-25,
adjusting the temperatures on the first heating plate and the second heating plate in the heating unit for a period of time by the temperature control unit, so that the first heating zone, the second heating zone and the third heating zone are respectively stable at temperatures T1, T2 and T3,
pumping continuously a sample to be tested into the inlet port of the microchannel of the microfluidic substrate for a period of time by the flow control unit, and detecting with the detection unit, and
analyzing with an analysis unit.

Embodiment 27 provides the PCR detection method of Embodiment 26, wherein the spacing d is in the range of 1 to 10 mm.

Embodiment 28 provides the PCR detection method of Embodiment 26, wherein the spacing d is in the range of 1 to 6 mm for a salt-free sample.

Embodiment 29 provides the PCR detection method of Embodiment 26, wherein the spacing d is in the range of 6 to 10 mm for a salt-containing sample.

Embodiment 30 provides the PCR detection method of Embodiment 26, wherein the temperature of the first heating plate is controlled in the range of 80° C. to 100° C. when T1 is greater than T2.

Embodiment 31 provides the PCR detection method of Embodiment 26, wherein the temperature of the second heating plate is controlled in the range of 55° C. to 65° C. when T1 is greater than T2.

Embodiment 32 provides the PCR detection method of Embodiment 26, wherein the temperature of the first heating plate is controlled in the range of 80° C. to 90° C. and the temperature of the second heating plate is controlled in the range of 55° C. to 65° C. for a salt-free sample.

Embodiment 33 provides the PCR detection method of Embodiment 26, wherein the temperature of the first heating plate is controlled in the range of 90° C. to 100° C. and the temperature of the second heating plate is controlled in the range of 55° C. to 65° C. for a salt-containing sample.

The exemplary embodiments disclosed above are merely intended to illustrate the various utilities of this disclosure. It is understood that numerous modifications, variations and combinations of functional elements and features of the present disclosure are possible in light of the above teachings and, therefore, within the scope of the appended claims, the present disclosure may be practiced otherwise than as particularly disclosed and the principles of this disclosure may be extended easily with appropriate modifications to other applications.

All patents and publications are herein incorporated for reference to the same extent as if each individual publication was specifically and individually indicated to be incorporated by reference. It should be understood that although the present disclosure has been specifically disclosed by preferred embodiments and optional features, modification and variation of the concepts herein disclosed may be resorted by those skilled in the art, and that such modifications and variations are considered to be within the scope of this disclosure.

What is claimed is:

1. A PCR detection device comprising
a heating unit comprising a first heating plate and a second heating plate, wherein the first heating plate and the second heating plate are arranged to be parallel and juxtaposed with a spacing d,
a microfluidic substrate comprising a microchannel, wherein the microchannel is configured in the microfluidic substrate with a plurality of isometric parallel back-and-forth cycles, and
a biological detection chip loaded at the end of the microchannel of the microfluidic substrate in a fluid-communicable manner,
wherein the microfluidic substrate is mounted on the heating unit, and contacts with the first heating plate to form a first heating zone and contacts with the second heating plate to form a second heating zone,
wherein the first heating plate applies a first temperature T1 to heat the first heating zone, and the second heating plate applies a second temperature T2 to heat the second heating zone,
wherein when T1 is different from T2, the region above the spacing d in the microfluidic substrate forms a third heating zone, and a third temperature T3 formed in the third heating zone is between T1 and T2,
wherein each isometric parallel back-and-forth cycle in the microfluidic substrate passes through the first heating zone, the second heating zone and the third heating zone.

2. The PCR detection device of claim 1, wherein the microchannel of the microfluidic substrate comprises one inlet port and at least one outlet port.

3. The PCR detection device of claim 1, wherein the spacing d is in the range of 1 to 10 mm.

4. The PCR detection device of claim 1, wherein the spacing d is in the range of 1 to 6 mm for a salt-free sample.

5. The PCR detection device of claim 1, wherein the spacing d is in the range of 6 to 10 mm for a salt-containing sample.

6. The PCR detection device of claim 1, wherein the temperature of the first heating plate is controlled in the range of 80° C. to 100° C. and the temperature of the second heating plate is controlled in the range of 55° C. to 65° C. when T1 is greater than T2.

7. The PCR detection device of claim 1, wherein the temperature of the first heating plate is controlled in the range of 80° C. to 90° C. and the temperature of the second heating plate is controlled in the range of 55° C. to 65° C. for a salt-free sample.

8. The PCR detection device of claim 1, wherein the temperature of the first heating plate is controlled in the range of 90° C. to 100° C. and the temperature of the second heating plate is controlled in the range of 55° C. to 65° C. for a salt-containing sample.

9. A PCR detection system, comprising
a heating unit comprising a first heating plate and a second heating plate, wherein the first heating plate and the second heating plate are arranged to be parallel and juxtaposed with a spacing d,
a microfluidic substrate comprising a microchannel, wherein the microchannel is configured in the microfluidic substrate with a plurality of isometric parallel back-and-forth cycles, wherein the microfluidic substrate is mounted on the heating unit and contacts with the first heating plate to form a first heating zone and contacts with the second heating plate to form a second heating zone, wherein the first heating plate applies a first temperature T1 to heat the first heating zone, and the second heating plate applies a second temperature T2 to heat the second heating zone,
a biological detection chip loaded at the end of the microchannel of the microfluidic substrate in a fluid-communicable manner,
a flow control unit for controlling the flow rate of liquid in the microfluidic substrate,
a temperature control unit, which is electrically connected to the heating unit and respectively controls the heating temperatures of the first heating plate and the second heating plate, and
a detection unit for detecting the biological detection chip,
wherein when T1 is greater than T2, the region above the spacing d in the microfluidic substrate forms a third heating zone, and a third temperature T3 formed in the third heating zone is between T1 and T2.

10. The PCR detection system of claim 9, wherein the biological detection chip is a surface plasmon resonance chip.
